(19) United States
(12) Patent Application Publication
(10) Pub. No.: US 2002/0193723 A1
(43) Pub. Date: Dec. 19, 2002

(54) Compressible foam wound dressing

(76) Inventors: David B. Batdorf SR., Templeton, CA (US); Mun-Chung Yun, Paso Robles, CA (US)

Correspondence Address: Michael G. Petit, PO Box 91929, Santa Barbara, CA, 93190-1929, US

(21) Appl. No.: 09/881,510

(22) Filed: Jun. 13, 2001

(51) Int. Cl. A61F 13/00

(52) U.S. Cl. 602/48; 602/46

(57) ABSTRACT

A surgical wound dressing wherein an elastomeric sheet of cured closed cell silicone foam forms a support layer. A silicone gel is applied to one surface of the foam sheet to form a skin-facing layer. The resultant wound dressing may be stretched and placed upon a person's skin to overlie a wound and provide a fluid impermeable barrier between the wound and the external environment. In addition, the closed-cell foam may be elastically compressed against the skin to reduce swelling and protect the wound from mechanical trauma during the healing process.

### BACKGROUND OF THE INVENTION

1. Field of the Invention

The present invention relates generally to wound dressings and more particularly to compressible wound dressings.

2. Prior Art

Elastomeric wound dressings having a gel layer on a skin-facing surface thereof are well known in the art. Pocknell, in U.S. Pat. No. 4,991,574, discloses a wound dressing comprising a sheet of cured silicone elastomer having a layer of silicone gel applied to a skin-facing surface thereof. The wound dressing is suitable for covering areas of a persons skin subject to burn injury but has limited applicability as a compression dressing due to the noncompressible structure of the elastomeric support layer as well as the gel layer. An advantage of the Pocknell wound dressing is that it is substantially transparent; enabling the wound to be viewed without removing the dressing. Nevertheless, there are patients that would prefer not to view a wound during the healing process.

Edenbaum, in U.S. Pat. No. 4,733,659, discloses a bandage/wound covering comprising a liquid permeable elastomeric foam sheet wherein a porous layer of an adhesive is applied to the foam sheet to form a skin-facing surface of the bandage. The support sheet is a hydrophilic foam such as polyurethane. Due to the porous nature of both the foam supporting sheet and the adhesive layer, the bandage is unsuitable for providing a moisture barrier between a wound and the external environment.

Chen, in U.S. Pat. No. 3,972,328, discloses a three-layer bandage comprising a sheet of “semi-open cell” elastomeric foam interposed between a skin-facing absorbant adhesive layer and an outer layer of a water impervious flexible polymeric film. The wound dressing has the advantage of being compressible but the semi-open cell structure in the foam layer draws fluid from the adhesive layer and serves as a reservoir for such fluid. The hydrophilic adhesive layer is, in operation, in contact with the wound and the bandage may be unsuitable for covering a wound wherein either there is no fluid exudate or it is undesirable to withdraw such exudates from the wound.

There remains a need for an elastically extensible and compressible wound dressing having a hydrophobic skin-facing surface. Further, there remains a need for a wound dressing that satisfies the foregoing requirements, has a long shelf life and is inexpensive to produce.

### SUMMARY

It is a primary object of the invention to provide a wound dressing that is elastically extensible and may be applied directly to the skin to overlie a wound.

It is a further object of the invention to provide a wound dressing meeting the above objective and which is compressible.

It is yet another object of the invention to provide a wound dressing meeting the above objective and which is substantially opaque.

The features of the invention believed to be novel are set forth with particularity in the appended claims. However the invention itself, both as to organization and method of operation, together with further objects and advantages thereof may be best be understood by reference to the following description taken in conjunction with the accompanying drawings in which:

### BRIEF DESCRIPTION OF THE DRAWINGS

FIG. 1 is an enlarged perspective view of a portion of a wound dressing in accordance with the present invention.

FIG. 2 is a cross-sectional view of a wound dressing in accordance with the present invention taken along section line 2-2 of FIG. 1.

### DESCRIPTION OF THE PREFERRED EMBODIMENTS

Inasmuch as the wound dressing of the present invention is made entirely of silicone, it is instructive to briefly discuss the nature and general properties of such compounds. Silicones are a group of completely synthetic polymers containing the recurring group —Si—RR′—O—, wherein R and R′ is a radical such as an alkyl, aryl, phenyl or vinyl group. The simpler short chain silicones are oils of very low melting point, while at the other end of the scale of physical properties, long chain silicones may be highly crosslinked to form flexible and even rigid solids. Partially cross-linked silicone elastomers such as gels have physical properties that are intermediate between these two extremes. Crosslinked long-chain silicones are suitable for forming an elastic sheet. The addition of a blowing agent (i.e., a compound that produces a gas under the curing conditions such as sodium bicarbonate) to the silicone elastomer prior to crosslinking provides, upon curing, a compressible, elastic sheet of silicone foam. Silicone gels may be used to provide a coating for the skin-facing surface of a wound dressing. A layer of silicone gel is cohesive and is adhesive to skin but non-adherent to wounds.

In accordance with the present invention, a sheet of Mylar® coated with a suitable silicone release material is placed on a flat surface such as the bottom of a tray to completely cover the surface thereof. Uncured silicone elastomer such as MED-4820 or MED-4870 (NuSil Corp., Carpinteria, Calif.) containing a blowing agent such as sodium bicarbonate dispursed therein is then placed upon the coated surface of the sheet at one end of the tray. The blowing agent is selected such that the blowing agent will, under curing conditions, produce closed cells having a diameter of between 0.0005 and 0.005 inches. A knife edge, spaced from the bottom of the tray by 0.01-0.03 inches, is drawn over the bottom of the tray to evenly spread the elastomer to form a uniform layer of silicone on the release film. The tray is then transferred to an oven and heated to about 250° F., a temperature sufficient to activate the blowing agent, and then to about 400° F. to cure the foam elastomer. The durometer of the resultant foam sheet is preferably between 20 and 70. The tray is then cooled and removed from the oven.

A quantity of silicone gel such as, for example, MED-6340 (NuSil Corp.) is placed on the cooled, cured sheet of silicone foam and distributed over the surface of the foam sheeting with a knife edge to form a gel layer on the foam sheet having a uniform thickness in the range of between 0.01 and 0.10 inches. The gel layer is then cross-linked, as, for example, by heating, and covered with a sheet of release film and the laminate sheet removed from the tray and cut into wound dressings having the desired size. While the gel layer can be made in any desired thickness, a preferred range of thickness is 0.01-0.10 inch.

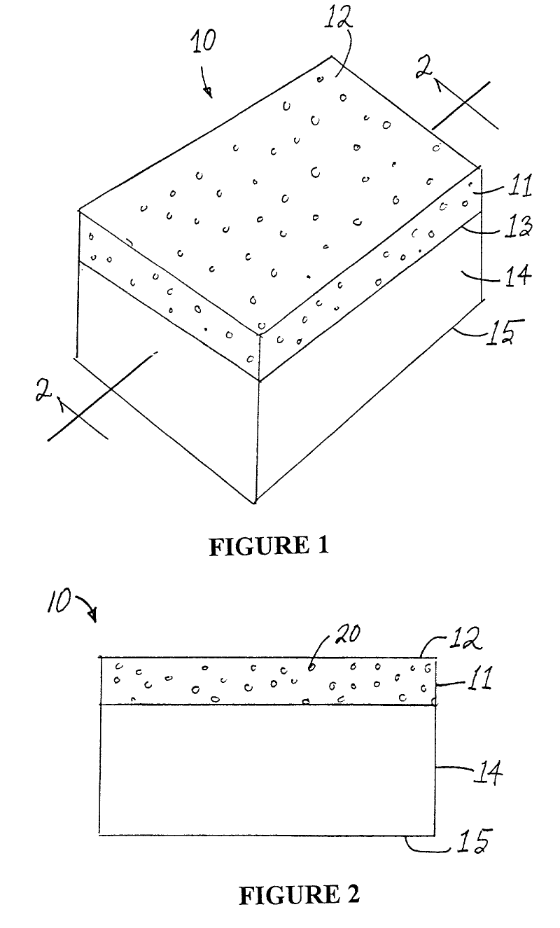

FIG. 1 is an enlarged perspective view of a portion of a wound dressing 10 in accordance with the present invention. The sheet of closed cell silicone foam 11 has an upper surface 12 and a lower surface 13. A hydrophobic gel layer 14, preferably of silicone gel, is adhered to the lower surface 13 of the foam sheet 11. FIG. 2 is a cross-sectional view of a wound dressing in accordance with the present invention taken along section line 2-2 of FIG. 1. The hydrophobic gel layer 14, adhered to the lower surface 13 of the foam, presents an imporforate, substantially impermeable surface that will adhere to skin but not to fluid exudates from a wound. The closed cells 20 dispursed throughout the foam 11 enable the foam to be elastically compressed while remaining fluid impermeable.

In use, the release film(s) are removed from the wound dressing and the gel layer is placed upon the skin to overlie the wound. The sheet may be stretched prior to such placement to compress the underlying skin. If a stretchable elastomer such as MED-4820 is used to form the foam sheet, the wound dressing will be very stretchable whereas if a higher durometer elastomer such as MED-4870 is used to form the foam sheet, the dressing will have less stretchability. The gel layer presents a substantially fluid impermeable non-toxic, biocompatible surface to the wound while adhering to the surrounding skin. The closed cell structure of the foam layer enables the wound dressing to be elastically compressed against the underlying tissue as desired.

While particular embodiments of the present invention have been illustrated and described, it would be obvious to those skilled in the art that various other changes and modifications can be made without departing from the spirit and scope of the invention. For example, the foam sheet and gel layer may by coextruded or made in a continuous process in any desired size. It is therefore intended to cover in the appended claims all such changes and modifications that are within the scope of this invention.

What we claim is:

1. A wound dressing operable for applying to the skin of a person to overly a wound, the wound dressing comprising an imperforate sheet of closed cell silicone foam having an upper surface and a lower surface and an adhesive layer of a hydrophobic gel adhered to said lower surface of said silicone foam.

2. A wound dressing in accordance with claim 1 wherein said hydrophobic gel is substantially impermeable to aqueous fluids.

3. A wound dressing in accordance with claim 1 wherein said hydrophobic gel is silicone gel.

4. A wound dressing in accordance with claim 2 wherein said hydrophobic gel is silicone gel.

5. A wound dressing in accordance with claim 1 wherein said imperforate sheet of closed cell silicone foam is comprised of a silicone elastomer having a durometer in the range of 20-70.
